(19) United States
(12) Patent Application Publication
(10) Pub. No.: US 2021/0101026 A1
(43) Pub. Date: Apr. 8, 2021

(54) LINAC QUALITY CONTROL DEVICE

(71) Applicant: Deniz ÇELIK, Istanbul (TR)

(72) Inventor: Deniz ÇELIK, Istanbul (TR)

(21) Appl. No.: 16/590,413

(22) Filed: Oct. 2, 2019

Related U.S. Application Data
(63) Continuation-in-part of application No. 16/320,491, filed on Jan. 25, 2019, now Pat. No. 10,918,889.

(51) Int. Cl. A61N 5/10; A61B 6/00

(52) U.S. Cl. CPC A61N 5/1075; A61N 2005/1085; A61B 6/583

(57) ABSTRACT

A quality control device which enables all the routine quality controls of linear particle accelerators (LINACs), which are used in radiation oncology, to be performed automatically. The quality control device includes a sensor panel having at least twenty one first optical sensors, at least two laser distance sensors, at least two g-sensors, at least one second optical sensor disposed at a depth of 1 mm from a surface of the sensor panel, and a 2 mm diameter hole located on the surface of the sensor panel above the at least one second sensor; a measurement panel including at least one linear light detector and at least one linear radiation detector for determining an isocenter that receives light and a possible cross-wire angle by performing a light area scanning; an inclinometer providing an angle correction by obtaining angle information from each position of the measurement panel; and a motorized system.

### CROSS REFERENCE TO THE RELATED APPLICATIONS

This application is a continuation-in-part application of U.S. patent application Ser. No. 16/320,491 filed on Jan. 25, 2019, which is is the national phase entry of International Application No. PCT/TR2017/050330, filed on Jul. 21, 2017, which is based upon and claims priority to Turkish Patent Application No. 2016/11175, filed on Aug. 9, 2016, the entire contents of which are incorporated herein by reference.

### TECHNICAL FIELD

The present invention relates to a quality control device which enables all the routine quality controls of linear particle accelerators (LINACs), which are used in radiation oncology, to be performed automatically.

### BACKGROUND

Today, quality control tests must be performed at certain points of installation and operation in order for the LINAC devices, which are one of the milestones in radiation oncology, to operate efficiently and accurately.

These tests may be listed as follows:
Controlling the collimator angle indicator,
Controlling the cross-wire stability upon collimator axis rotation,
Conformity of cross-wire axis and radiation field axis,
Controlling the gantry angle indicator,
Isocenter control,
Laser control,
Controlling the optical distance indicator,
Dependence of the optical distance indicator on the gantry angle,
Controlling the indicator of field sizes,
Controlling the conformity of the light-irradiation field,
Arc therapy angle control,

In addition to these tests, the measurements between the treatment table on which the LINAC device is used and the device itself must be conducted.

These tests, on the other hand, may be listed as follows:
Controlling the angle indicator with the isocentric rotation of the treatment table,
Conformity of the isocenter with the rotational movement of the treatment table,
Controlling the change in the conformity of the isocenter with the rotational movement of the treatment table depending on the weight,
Controlling the parallelism of the collimator axis with the vertical movement of the table,
Controlling the change in the parallelism of the collimator axis with the vertical movement of the table depending on the weight,
Asymmetrical collimator control,
Mechanical position test of kilovolt source,
Mechanical position test of kilovolt detector.

The mechanical quality control tests conducted using the quality control equipment in the state of the art are visual measurements performed by the user and the results of such measurement may vary from one user to another due to the human factor. Some tests include measurements that are marked on graph paper by pens and the deviation is detected visually by the user in such measurement. This, however, affects not only the accuracy but also the repeatability of the test.

For other tests, different measurement equipment is employed, and so it takes a longer time to complete all the tests.

The equipment used in the state of the art for carrying out such tests is: graph papers, needles, meters, calibrated rods of varying lengths, and digital levels.

The way of conducting the tests mentioned above using the equipment within the state of the art and the resulting technical problems are described hereinafter.
The collimator angle indicator is controlled using a digital level while the gantry is 90°. In this case, additional uncertainty occurs depending on whether the digital level is located properly. Moreover, the deviations resulting from the digital level may also have an impact on the accuracy of the measurement.
Controlling the cross-wire stability upon collimator axis rotation is a test conducted for detecting the deviation from the center of the treatment depending on the collimator rotation. A dot is made on a paper and the deviation from this dot is tried to be detected from different collimator angles. It is a test in which the user can detect such deviations only by visual inspection, as a result of which the accuracy, precision and speed of the measurements may vary from one user to another.
Controlling the conformity of cross-wire axis and radiation field axis refers to the detection of the conformity of the radiation field axis as obtained by irradiation at different collimator angles when the gantry is 0°. However, several drawings need to be made on the image obtained by irradiation in order to define the result of this test; therefore, the result of this measurement is dependent on the drawing skill of the user.
The gantry angle indicator is controlled using a digital level while the gantry is 0°, 90°, 180° and 270°. Here, the technical problems are the same as those experienced in the test of controlling the collimator angle indicator.
For isocenter control, the central image reflected on a rod attached at the end of the table is used in order to detect the deviation from the center in gantry angles. Here, the main problem is that it is challenging to detect the central projection and that the precision is entirely dependent on the user.
The method and technical problems regarding laser control are the same as in the isocenter control test.
For controlling the optical distance indicator, measurement is made using a rod attached to the head of the LINAC device. The treatment table is lifted until it contacts with the rod and measurements are taken from this distance. The main problem here is that the rod has a telescopic mechanism in order that its end will not damage the table. Although this prevents the rod from damaging the table, it directly affects the result of the micron-level measurement.
The method and technical problems regarding the detection of the deviation of the optical distance indicator depending on the gantry angle are again the same as in the isocenter control test.
In this test performed for controlling the indicator of field sizes, on the other hand, different field sizes are opened and then it is controlled whether the field sizes are opened correctly or not. The field sizes opened for this test are detected visually by the user using graph paper. The measurement results are directly dependent on the human factor, and hence the user's precision.
In the test of controlling the conformity of the light-irradiation field, the conformity of the field, which is considered to be opened correctly in physical terms, with the radiation field is detected. During this test, it is important to accurately locate the film which detects the radiation field since the lower or higher position of the film changes the size of the field being measured.
In this test conducted for the angle control of the arc therapy, the gantry is moved at certain angles and the deviation at these angles is detected. The problem in this test is the same as that experienced while controlling the collimator angle indicator. Further, it is another important technical problem that measurement cannot be made at intermediate angles. For example, only the deviation at 0° and 90° can be detected in case of 0° and 90° arc control. As a result, it is not possible to detect the deviations and sizes of deviations at the angles at interval values.
The conformity of the isocenter with the rotational movement of the treatment table is the test conducted for detecting the deviation from the isocenter depending on the rotational movement of the treatment table and the technical problem and the uncertainty in this test are the same as in the test of controlling the cross-wire stability upon collimator axis rotation.
The test for controlling the change in the conformity of the isocenter with the rotational movement of the treatment table depending on the weight, on the other hand, is a different version of the test of controlling the conformity of the isocenter with the rotational movement of the treatment table in which a weight is placed on the table during the test. The reason for placing a weight on the table is to simulate the weight of the patient to lie on the table during the therapy. Similar technical problems are experienced here.
Controlling the parallelism of the collimator axis with the vertical movement of the table is the test used for detecting the deviation from the isocenter based on the vertical movement of the table. The technical problem and the uncertainty in this test are the same as in the test of controlling the cross-wire stability upon collimator axis rotation.
The test for controlling the change in the parallelism of the collimator axis with the vertical movement of the table depending on the weight, on the other hand, is a different version of the test of controlling the parallelism of the collimator axis with the vertical movement of the table in which a weight is placed on the table during the test. The reason for placing a weight on the table is to simulate the weight of the patient to lie on the table during the therapy. Similar technical problems are experienced here.
It is determined in this test, which is performed for controlling the asymmetrical field, whether a coincidence resulting from field combination is present or not. The technical problem and the uncertainty in this test are the same as in the test of controlling the conformity of the light-irradiation field.
In the mechanical position test of the kilovolt source, ruler measurement is made for confirming the position of the source at distances of 80, 90 and 100 cm. The main problem in measurements is the precision and accuracy errors resulting from the ruler-based measurement.
In the mechanical position test of the kilovolt source detector, ruler measurement is made for confirming the position of the source at distances of −30, −50 and −75 cm. The main problem in measurements is the precision and accuracy errors resulting from the ruler-based measurement.

Apart from the manual tests in the state of the art which are described in detail above, there also exist automated systems which have been developed. The European Patent Application No. EP2701802 and the U.S. Pat. Nos. 8,845,191, 9,283,405, 6,614,036, 6,626,569 and 7,476,867 may be given as examples to these systems.

In order to clearly describe the aforementioned procedures, the explanations as to the components used in the related technical field are made below:
Gantry: It is a treatment head which may be circularly rotated around the patient and in which electrons and x-rays are generated.
Collimator: It is a type of protection block which is disposed in the gantry and used for shaping the therapy area by filtering x-rays.
LINAC: Known as Linear Particle Accelerator (LINAC), this device generates high energy x-rays and electrons. The electrons ejected from the metal target under high voltage are accelerated within the electromagnetic field such that they will have a higher energy. While the high energy electron beam can be used in surface tumors, the high energy x-rays obtained as a result of making them hit a target are used in the treatment of deeply located tumors.
Isocenter (cross-wire): The point of treatment center in which the rotational axes of the gantry, collimator and couch of the LINAC device coincide.
Arc Therapy: A radiotherapy method in which the LINAC device operates by rotating around the patient and the shape and intensity of the radiation beams are constantly changed.
ODI (Optical Distance Indicator): It is an optical distance indicator which allows digital display of the distance with respect to the source in LINAC device.

### SUMMARY

The most important and common technical problem in the tests conducted with the equipment in the state of the art which have been described above in detail is that they are vulnerable to faults resulting from human errors because they are performed manually.

In addition to this, the so-called IsoCheck, a measuring instrument, is used in some of the mechanical measurements. Prior to starting measurements using this instrument, it is required to be adjusted using the digital level such that it will be properly positioned and be parallel with respect to the gantry; otherwise, uncertainty may occur in the measurements.

Not only making these adjustments is time-consuming but also positioning of the device may differ from one user to another. The device according to the invention, however, automatically adjusts its center and parallelism with respect to the gantry, thereby not only saving on time but eliminating the uncertainty based on the installation of the device as well.

Moreover, all of these tests are performed automatically thanks to the quality control device according to the invention and measurement results are obtained with more precision and accuracy when compared to manual methods.

The tests carried out using a digital level in the state of the art are performed using 2 different laser distance measuring systems and G-sensor (gyrosensor/gyroscope) in the device according to the invention and the angular deviations in the gantry can be detected with high precision.

The tests conducted by a dot made on the paper in the state of the art are performed using optical sensors (photodiodes) in the device according to the invention and the movements of the table, collimator and gantry are digitally monitored, thereby detecting the deviations with high precision.

Thanks to the device according to the invention, the tests performed by means of a radiographic film positioned using LINAC variants (laser, optical distance indicator, isocenter) in the state of the art are performed using a radiographic film properly positioned by means of laser distance sensors instead.

Rather than the detection made by the central image reflected on a rod which is attached to an end of the table with a view to detect the deviations from the center at different gantry angles in the state of the art, the deviations are digitally detected with high precision using optical sensors (photodiodes) with the device according to the invention.

The uncertainty regarding the test of controlling the optical distance indicator, the mechanical position test of the kilovolt source and mechanical position test of the kilovolt detector can be eliminated using a laser distance meter in the device according to the invention.

### BRIEF DESCRIPTION OF THE DRAWINGS

The drawings illustrating the LINAC quality control device developed with the present invention are given below for a better understanding of the invention.

FIG. 1A shows a perspective view of the LINAC device and the treatment table of the LINAC device.

FIG. 1B shows a perspective view of the treatment head at 90°.

FIG. 1C shows a perspective view of the treatment head at 180°.

FIG. 1D shows a perspective view of the treatment head at 270°.

FIG. 1E shows a perspective view of the treatment table at 90°.

FIG. 1F shows a perspective view of the treatment table at −90°.

FIG. 2 shows a perspective view of the LINAC quality control device according to the invention.

FIG. 3 shows a top view of the sensor panel in the LINAC quality control device according to the invention.

FIG. 4 shows perspective view of the motorized systems moving the sensor panel in the LINAC quality control device according to the invention.

FIG. 5 shows a perspective view of the optical sensor system which is located at a certain depth from the surface of the sensor panel in the LINAC quality control device according to the invention.

FIG. 6A shows a top left view of a UFC device.

FIG. 6B shows a top view of the UFC device.

FIG. 6C shows a top right view of the UFC device.

FIG. 6D shows a left view of the UFC device.

FIG. 6E shows a back view of the UFC device.

FIG. 6F shows a right view of the UFC device.

FIG. 6G shows a bottom left view of the UFC device.

FIG. 6H shows a bottom view of the UFC device.

FIG. 6I shows a bottom right view of the UFC device.

FIG. 7A shows a top left view of a measurement panel.

FIG. 7B shows a top view of the measurement panel.

FIG. 7C shows a top right view of the measurement panel.

FIG. 7D shows a left view of the measurement panel.

FIG. 7E shows a back view of the measurement panel.

FIG. 7F shows a right view of the measurement panel.

FIG. 8 shows positions and locations of photodiodes.

FIG. 9 shows Detector Positions.

FIG. 10 shows X-Ray Detector Position and Size (Measurement Panel Cross Section).

FIG. 11 shows X-Ray Detector Position (Measurement Panel Vertical Section).

FIG. 12 shows directions of UFC Measurement Panel Movements.

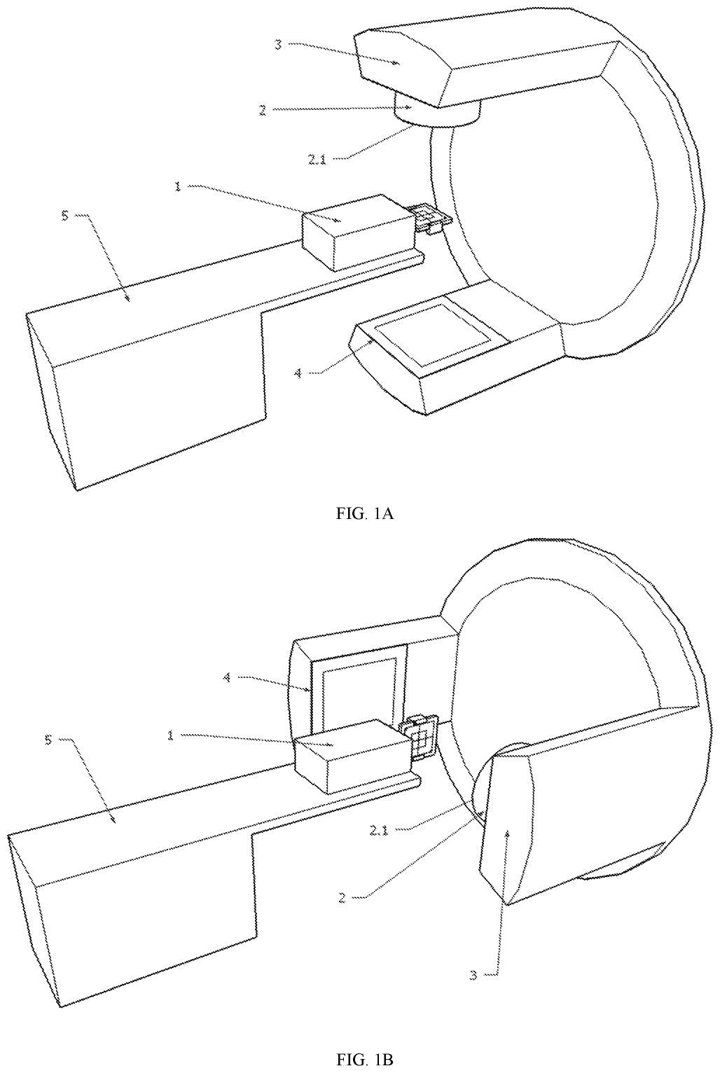

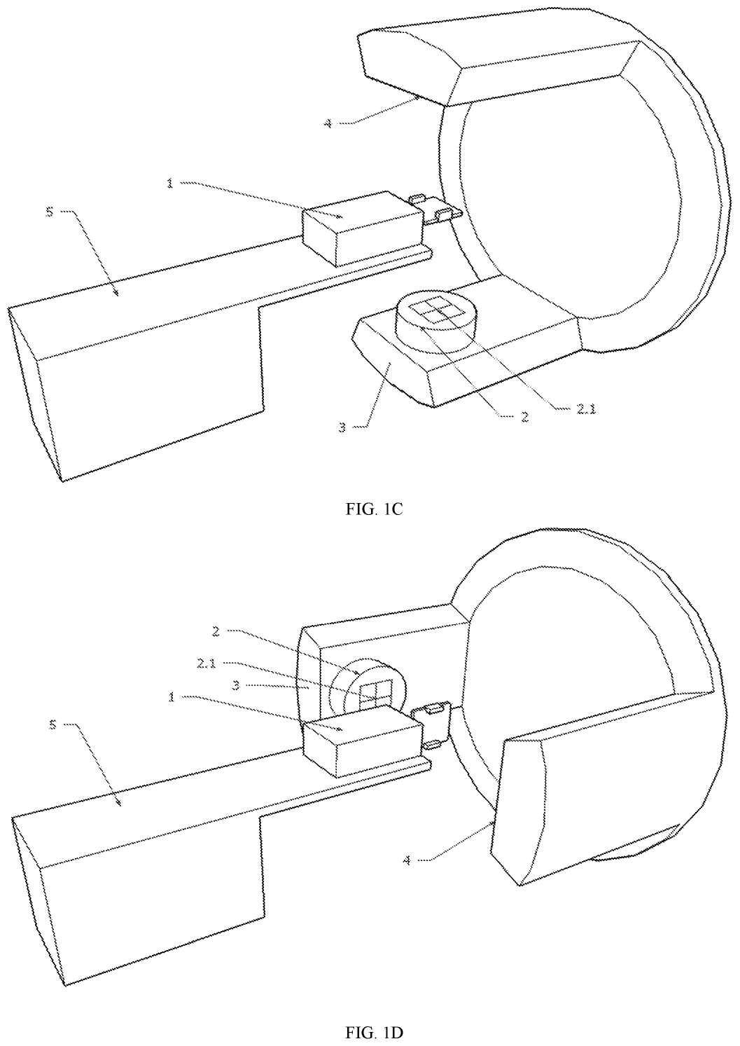

### DESCRIPTION OF THE PART REFERENCES

The parts/portions/components which are shown in the drawings illustrating the LINAC quality control device developed with the present invention for a better understanding of the invention are enumerated individually and the reference numbers corresponding thereto are given below.
1. LINAC quality control device
2. Collimator
2.1 Cross-wire (isocenter) Central axis of the area in which treatment will be made
3. LINAC treatment head rotatable 360° around the patient
4. Portal imaging system which allows taking images of the patient before and during the treatment
5. Treatment table capable of moving at 6 axes (back-and-forth, up-and-down, horizontal, rotational, angular, pitch) on which the patient lies during the treatment
6. Sensor panel.
6.1. Back-and-forth movement of the panel
6.2. Horizontal movement of the panel
6.3. Rotational movement of the panel
7. Motorized system capable of moving the sensor panel at 3 axes
8. Impact-resistant casing of the LINAC quality control device
9.1. Optical sensor 1
9.2. Optical sensor 2
9.3. Optical sensor 3
9.4. Optical sensor 4
9.5. Optical sensor 5
9.6. Optical sensor 6
9.7. Optical sensor 7
9.8. Optical sensor 8
9.9. Optical sensor 9
9.10. Optical sensor 10
9.11. Central optical sensor 11
9.12. Optical sensor 12
9.13. Optical sensor 13
9.14. Optical sensor 14
9.15. Optical sensor 15
9.16. Optical sensor 16
9.17. Optical sensor 17
9.18. Optical sensor 18
9.19. Optical sensor 19
9.20. Optical sensor 20
9.21. Optical sensor 21
10.1. Laser distance sensor 1
10.2. Laser distance sensor 2
11.1. G-sensor 1
11.2. G-sensor 2
12.1. Rotational movement motor
12.2. Horizontal movement motor
12.3. Back-and-forth movement motor
13.1. Back-and-forth movement gear
13.2. Horizontal movement gear
14.1. Back-and-forth movement motor bearing system
14.2. Horizontal movement motor bearing system
15.1. Linear bearing system accommodating the horizontal movement motor
15.2. Linear bearing system accommodating the back-and-forth movement motor
16. Belt and pulley mechanism of rotational movement
17. Optical sensor
18. 2 mm diameter hole opened on the optical sensor.
19.1 UFC Device Top Left View
19.2 UFC Device Top View
19.3 UFC Device Top Right View
19.4 UFC Device Left View
19.5 UFC Device Back View
19.6 UFC Device Right View
19.7 UFC Device Top Left View
19.8 UFC Device Bottom View
19.9 UFC Device Bottom Right View
20 Measurement Panel
20.1 Measurement Panel Top Left View
20.2 Measurement Panel Top View
20.3 Measurement Panel Top Right View
20.4 Measurement Panel Left View
20.5 Measurement Panel Back View
20.6 Measurement Panel Right View
21.1 Linear Light Photodiodes
21.2 Center top Photodiode
21.3 Center Left Photodiode
21.4 Center Photodiode
21.5 Center Right Photodiode
21.6 Center Bottom Photodiode
21.7 Inclinometer
22 Radiation Detector
A. Outward Movement of the Measurement Panel
B. Inward Movement of the Measurement Panel

### DETAILED DESCRIPTION OF THE EMBODIMENTS

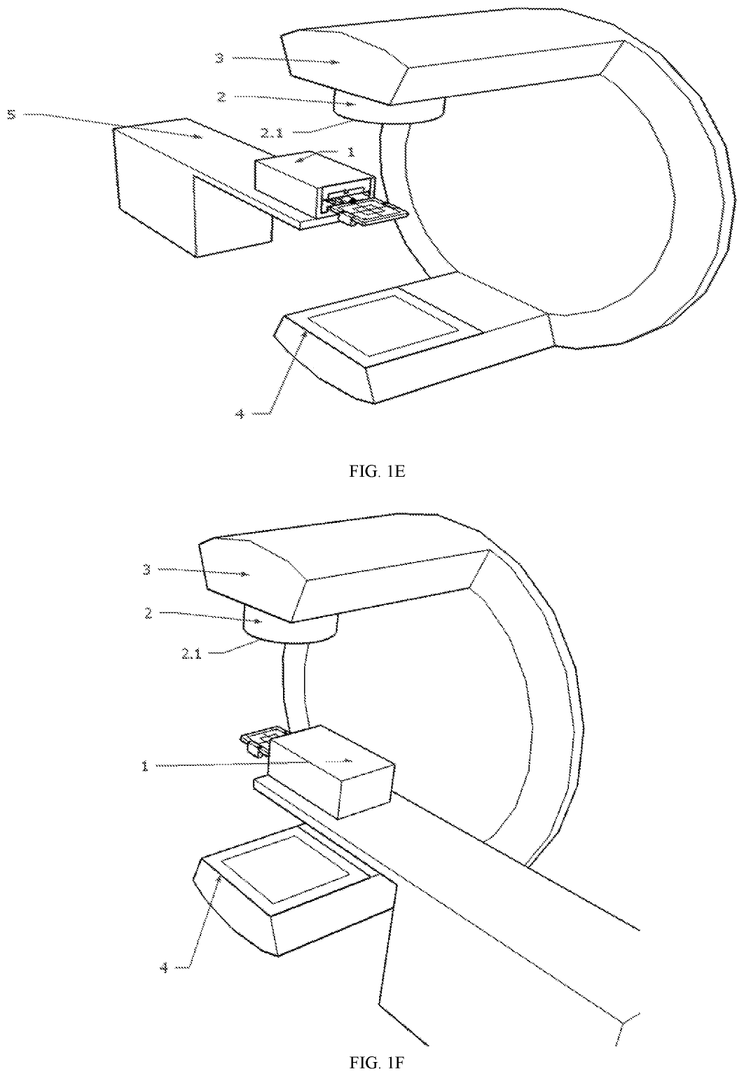

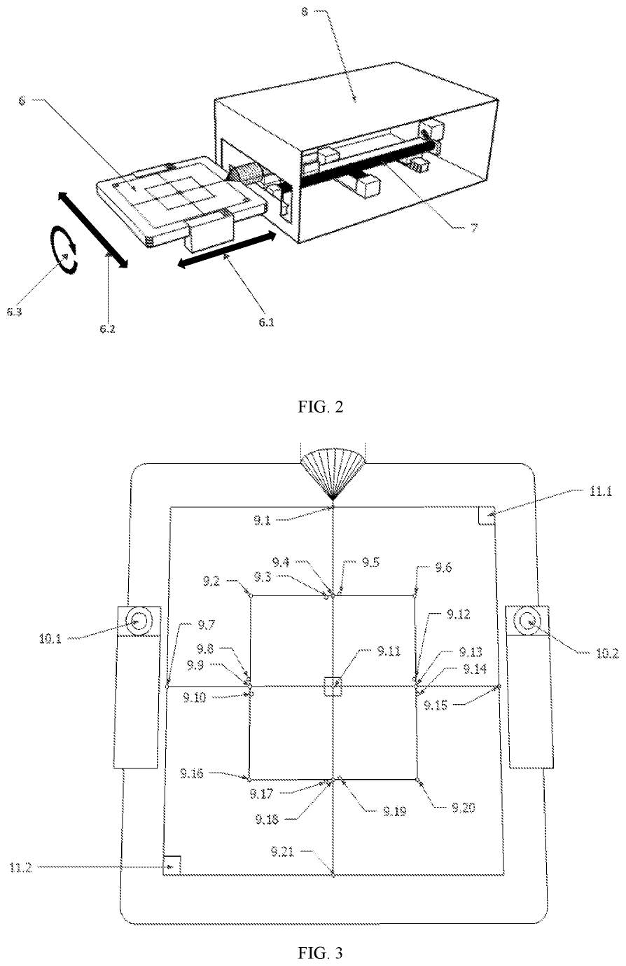

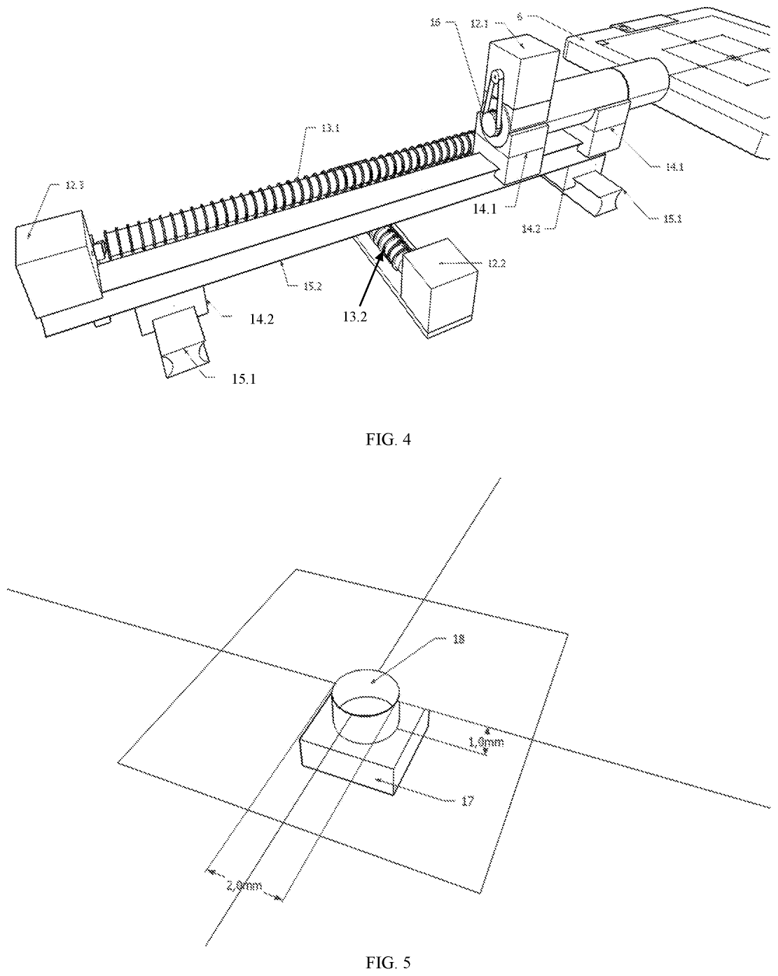

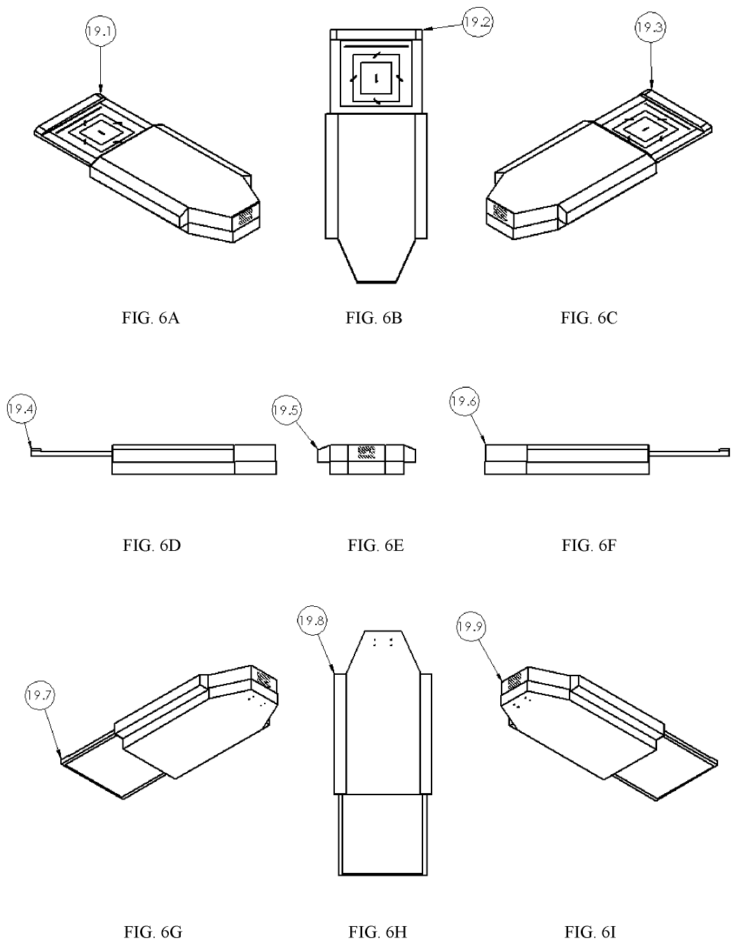

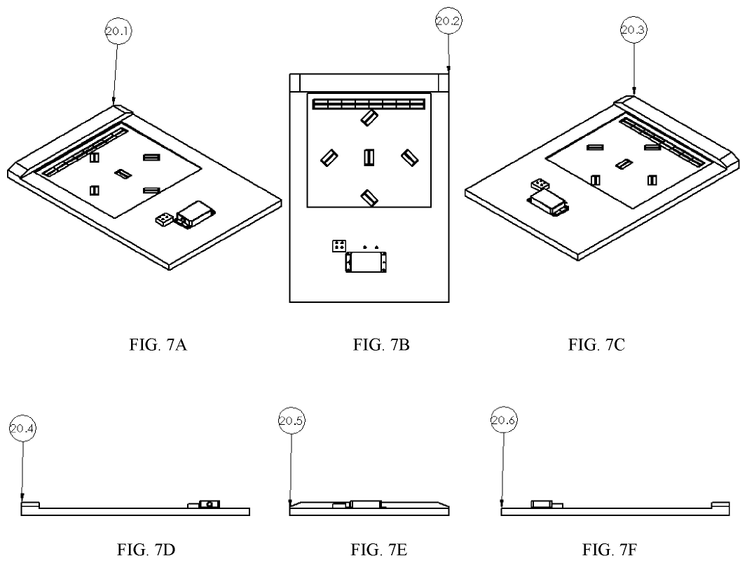

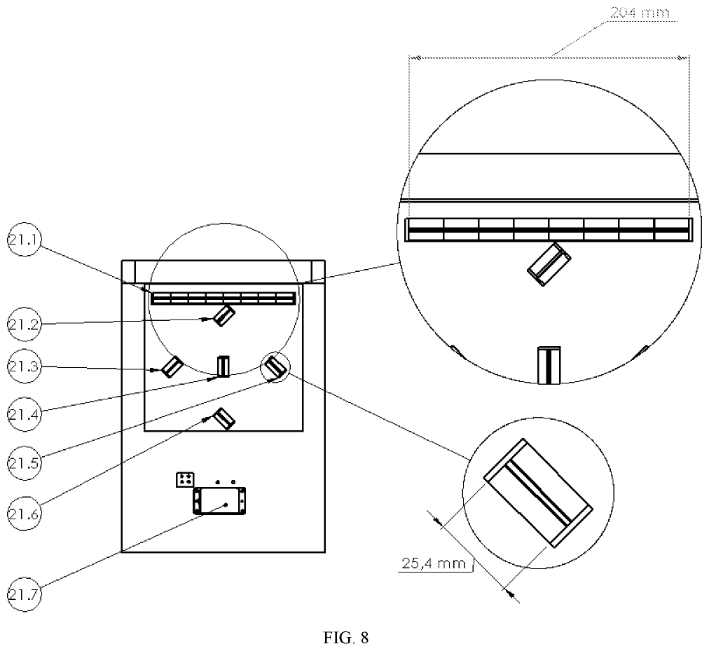

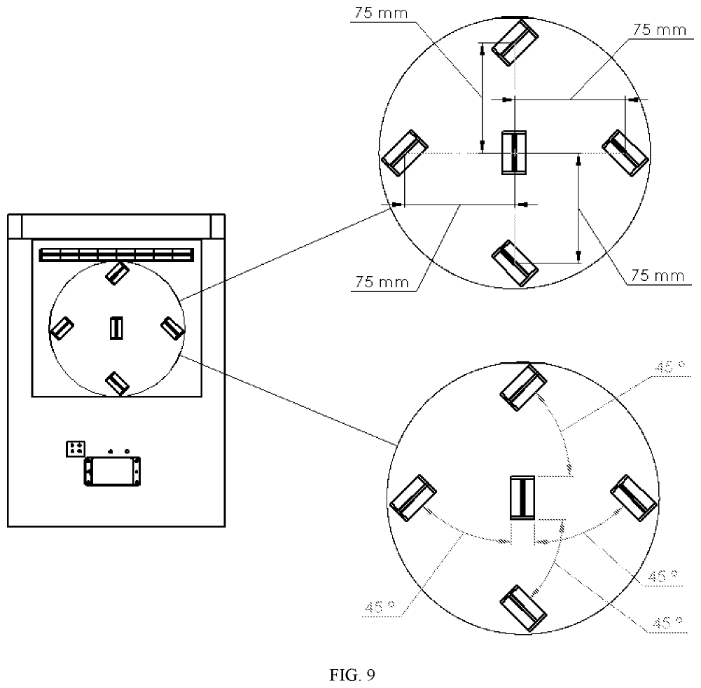

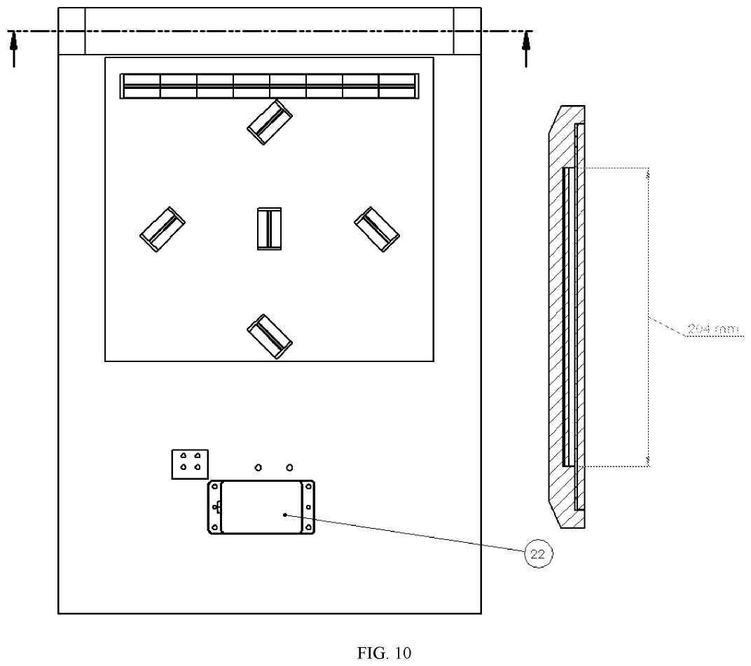

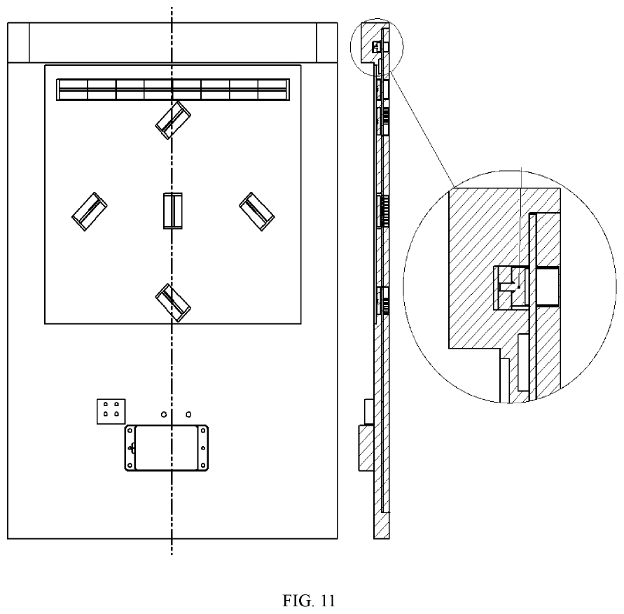

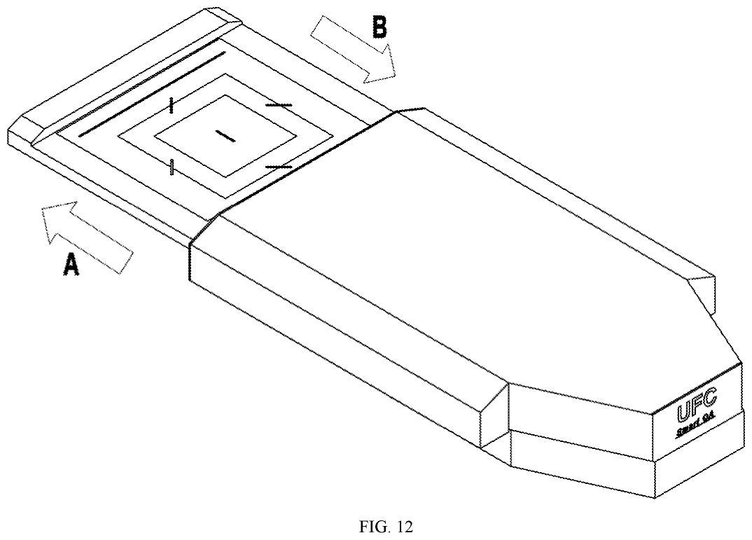

Sensor Panel (6): The sensor panel (6) which may be positioned in the casing (8) of the quality control device (1) when not in use and which, during the controlling process, can be made to assume its operational position by protruding from the end portion of the casing (8), is provided thereon with the following such that the required measurements will be performed:
At least 21 optical sensors (9.1-9.21),
At least 2 laser distance sensors (10.1 and 10.2),
At least 2 G-sensors (11.1 and 11.2),
At least one optical sensor (17) which is located at a depth of 1 mm from the surface of the panel (6), and
A 2 mm hole (18) made in the portion of the panel (6) surface coming over the optical sensor (17) in order to provide the viewpoint of the sensor (17).
   All of said sensors are disposed on one surface of the panel (6) and the panel (6) surface on which such sensors are located is entirely flat.
   The positioning of the sensors is as shown in FIGS. 3 and 5 in the primary embodiment of the invention; however, they may be positioned differently in different embodiments of the invention.
   Thanks to the motorized system (7) to which the sensor panel (6) is connected, the latter is capable of moving at 3 axes: back-and-forth (6.1), horizontal (6.2) and rotational (6.3).
   Motorized System (7): The motorized system (7), which is the mechanism that enables the sensor panel (6) to move at 3 axes, i.e. back-and-forth, horizontal and rotational axes, further allows the panel (6) to be introduced into and protrude from the casing (8) (6.1), and when in protruded position, to move in horizontal direction (6.2) and rotationally (6.3).
   The motorized system (7) within the casing (8) comprises:
At least 1 rotational movement motor (12.1) allowing the rotational movement of the panel (6),
At least 1 horizontal movement motor (12.2) allowing the movement of the panel (6) in horizontal plane,
At least 1 back-and-forth movement motor (12.3) allowing the back-and-forth movement of the panel (6),
At least 1 back-and-forth movement gear (13.1) transferring the drive of the back-and-forth movement motor (12.3) to the panel (6),
At least 1 horizontal movement gear (13.2) transferring the drive of the horizontal movement motor (12.2) to the panel (6),
Back-and-forth movement motor bearing system (14.1) accommodating the back-and-forth movement motor (12.3),
Horizontal movement motor bearing system (14.2) accommodating the horizontal movement motor (12.2),
Horizontal movement motor linear bearing system (15.1) accommodating the horizontal movement motor (12.2),
Back-and-forth movement motor linear bearing system (15.2) accommodating the back-and-forth movement motor (12.3), and
At least one belt and pulley mechanism (16) of rotational movement which transfers the drive of the rotational movement motor (12.1) to the panel (6).
   Casing (8): It serves as a shell which provides protection against impacts, in which the motorized system (7) is disposed and the sensor panel (6), when not in use, is positioned.
   In the primary embodiment of the invention, the casing (8) is made of any type of metal alloy, e.g. aluminium or steel; furthermore, it may as well be made of polymer material according to the application area.

Film for dosimetric tests, 2 dimensional measurement apparatus and water phantom equipment such as the field testing equipment that have been drawn and can be rotated by hand are used in the invention. The position of the equipment is determined by the user and the equipment is aligned and usually measurement is carried out by the sight of the user. The equipment used in mechanical quality controls of medical LINAC devices, are positioned by the user and the alignment is performed again by the user. Measurements carried out with said equipment can lead to uncertainty depending on the positioning of the user. Similarly the measurements carried out by such equipment are precise such that they can be determined by the eye of the user and their repeatability is low. As the measurements carried out during the tests are carried out by the sight of the user, the deviation amounts that occur at the end of the test are recorded by the user. Due to this reason measurements can be taken only at certain angles and distances.

Although equipment used for dosimetric quality control testing are digital, the initial positioning and alignment of said equipment are again performed by the user. Similar to mechanical testing uncertainty is again possible as mistakes can be made at the initial point of measurement. As the positions of detectors on the dosimetric quality control equipment are stable (except the water phantom) the resolution of the measurements carried out with these equipment are dependent on the number and size of the detectors used by the manufacturer. The quality control equipment (UFC) subject to the invention, first of all determines the light area isocenter and the possible cross-wire angle by scanning the light area with the measurement panel (20) after it is set on the treatment table. At the same time angle correction is carried out by using a software after obtaining the angle information of the measurement panel at each position of the measurement panel with the high precision inclinometer (21.7) located thereon. By this means, the UFC device becomes independent from the user during initial positioning and inclination and the uncertainty due to these actions are eliminated.

As a result of the scan carried out by means of the linear light photodiodes (21.1) that have been disposed on the measurement panel, light area and cross-wire position and sizes can be determined. By means of the light photodiodes (21.1, 21.2. 21.3, 21.4, 21.5, 21.6) positioned at the centre, the cross-wire position can be determined. By using these two detector groups at the same time, isocenter determination can be carried out for each angle of the cross-wire. Thereby measurements which can be performed only at certain angles using other equipments can be carried out in all angles, continuously.

By means of the linear radiation detectors (22) disposed in the measurement panel the position and size of the radiation field can be determined. The UFC device can, not only conduct dosimetric tests such as symmetry and smoothness tests using the field data red by these detectors, it can also compare the positions of the light field and the radiation area in comparison to each other.

The UFC device collects data with a scanning method by moving the linearly aligned detectors in order to read the light area and radiation areas. By means of the motor that can rotate angularly with high precision and the encoder that controls motor rotation, the positions on which position the measurement panel can carry out measurement shall be able to be adjusted by software. By this means the field reading resolution of the UFC device can be changed according to the preference of the user. The medical LINAC quality control equipment that is to be produced (UFC), can perform the task of several different measurement equipments used for mechanical and dosimetric tests on its own, with higher accuracy, higher precision and higher repeatability. The medical LINAC users that do not need to use a different equipment for each test, can perform quality controls in shorter periods of time and can store the results of the measurements carried out digitally.

By means of conducting the measurements carried out with a UFC device with higher precision, higher repeatability and higher accuracy, and by determining the possible medical LINAC device deviations easily and with precision, the patients will be able to receive accurate and precise treatment.

By being able to measure and compare the light area and radiation area with a single measurement system it is easier to determine the possible deviations in treatment techniques in which the light area is used as reference and to carry out the determinations of these deviations with higher accuracy and precision.

The tests that can be carried out only from the outside of the treatment room using radiation field, can be conducted from inside the treatment room using light area by being able to measure and compare the light area and radiation area with higher accuracy and precision. Due to this feature, the obligation of the service engineers to leave the treatment room during correction of possible deviations in the medical LINAC device is eliminated.

By means of the motion range of the adjustable measurement panel, the UFC device can reach very high resolutions, can be used for small area dosimetry which is challenging for the physicists.

Using the light detectors positioned at the center, the UFC device which can carry out cross-wire tracking, is able to receive the cross-wire position and angle not only at certain angles but continuously. As a result, it can determine the angle, the amount and the direction of the deviation. This information shows the exact position of the error for the service engineer who corrects the deviation that may occur in the medical LINAC device and it makes it much easier for the error to be corrected. The UFC device reads the field data by moving the light and radiation detectors positioned linearly thereon. By this means the resolution of the measurements carried out can be adjusted according to the preference of the user. Also, by means of this mobility, the number of detectors to be used in the measurement panel is significantly reduced and it has been enabled for the light and radiation detectors to be disposed on a single measurement panel.

By the aid of the high resolution inclinometer positioned on the measurement panel, the UFC device determined the angular errors of the measurement panel and it corrects them with software. The high resolution encoder connected to the motor system which moves the measurement panel determines the accuracy of the position of the measurement panel. Due to these features the UFC device can continuously test the accuracy of its own measurements.

The light detectors positioned at the center of the measurement panel, determine the cross-wire position and angle with high precision. By using the linear light detectors and the center detectors at the same time, the cross-wire position and each angle of the cross-wire can be read.

What is claimed is:

1. A quality control device for automatically performing all routine mechanical quality controls of linear particle accelerators (LINC) used in radiation oncology, comprising:
a sensor panel positioned inside a case when not being used and enters an operational mode by exiting out of an end section of the case during a control process;
wherein, the sensor panel comprises at least twenty one first optical sensors, at least two laser distance sensors, at least two g-sensors, at least one second optical sensor disposed at a depth of 1 mm from a surface of the sensor panel, and a 2 mm diameter hole opened at a portion located on the surface of the sensor panel above a section where the at least one second sensor is disposed in order to provide a clear field of view of the at least one second optical sensor;
a measurement panel comprising at least one linear light detector and at least one linear radiation detector for determining an isocenter that receives light and a possible cross-wire angle by performing a light area scanning,
an inclinometer providing an angle correction by obtaining angle information from each position of the measurement panel; and
a motorized system configured to move the sensor panel located inside the case along three axes; the movement along three axes being back- and forth, horizontal and rotational;
wherein, the motorized system comprises at least one rotational movement motor providing the rotational movement of the sensor panel, at least one horizontal movement motor providing the horizontal movement of the sensor panel on a horizontal plane, at least one back and forth movement motor providing the back and forth movement of the sensor panel, at least one back and forth movement gear transferring a drive of the back and forth movement motor to the sensor panel, at least one horizontal movement gear transferring a drive of the horizontal movement motor to the sensor panel, a back-and-forth movement motor bearing system accommodating the back-and-forth movement motor, a horizontal movement motor bearing system accommodating the horizontal movement motor, a horizontal movement motor linear bearing system accommodating the horizontal movement motor), a back-and-forth movement motor linear bearing system accommodating the back-and-forth movement motor, and at least one belt and pulley mechanism of rotational movement for transferring a drive of the rotational movement motor to the sensor panel;
wherein, the case serves as a shell providing protection against impacts and the motorized system is disposed in the case.
